(19) United States
(12) Patent Application Publication
(10) Pub. No.: US 2010/0136681 A1
(43) Pub. Date: Jun. 3, 2010

(54) CELLULAR COMPOSITIONS FOR THE TREATMENT OF KIDNEY DISEASE AND USES THEREOF

(75) Inventor: M. Amin Arnaout, Chestnut Hill, MA (US)

Correspondence Address: EDWARDS ANGELL PALMER & DODGE LLP, P.O. BOX 55874, BOSTON, MA, 02205, US

(73) Assignee: THE GENERAL HOSPITAL CORPORATION, Boston, MA (US)

(21) Appl. No.: 12/594,079

(22) PCT Filed: Mar. 18, 2008
(86) PCT No.: PCT/US08/03534

§ 371 Date: Jan. 27, 2010

Related U.S. Application Data
(60) Provisional application No. 60/921,658, filed on Apr. 2, 2007.

(51) Int. Cl. C12N 5/00

(52) U.S. Cl. 435/325

(57) ABSTRACT

As described in more detail below, the present invention provides cellular compositions for the treatment or prevention of kidney disease. The invention is based, at least in part, on the discovery of a population of cortical peritubular Flk1- and Seal-expressing kidney cells having a high tubulogenic potential.

### RELATED APPLICATION

This application claims priority to U.S. provisional application Ser. No. 60/921,658, filed Apr. 2, 2007, the entire disclosure of which is incorporated herein by this reference.

### BACKGROUND OF THE INVENTION

Acute and chronic kidney disease are common clinical problems with increasing incidence, serious consequences, and heavy financial strain. Approximately 5% of adults over 20 years of age have chronic kidney disease. Despite decades of basic research and important advances in patient care, the mortality rate of patients with acute renal injury is unacceptably high, between 30-80% in the intensive care setting. Ischemic or toxic injury to tubular epithelium is the major cause of acute renal failure, affecting ˜7% of hospitalized patients, and dialysis techniques, such as continuous renal replacement therapy, have had no significant impact on overall mortality. Unable to make new nephrons, the adult kidney responds to acute injury by the dedifferentiation and proliferation of surviving tubular cells adjacent to areas of damaged epithelium. A major limitation to such healing is the requirement for a critical number of surviving tubular cells to restore structural integrity of nephrons.

### SUMMARY OF THE INVENTION

As described below, the present invention features cellular compositions for the treatment or prevention of kidney disease.

In one aspect, the invention features an isolated cell having tubulogenic potential, where the cell is Flk1 positive Sca1 positive and CD34 negative.

In another aspect, the invention features an isolated cell having tubulogenic potential, where the cell is a kidney derived cell that is Flk1 positive Sca1 positive.

In various embodiments of the previous aspects, the cell is derived from adult or embryonic kidney. In other embodiments of these aspects, the cell fails to express a polypeptide or expresses reduced levels (e.g., 30%, 50%, 75%, 85%, or 95% less) of a polypeptide that is any one or more of c-kit, CD34, CD45, CD31, and cytokeratin relative to a reference. In other embodiments of these aspects, the cell is vimentin positive.

In another aspect, the invention features an isolated adult kidney cell having tubulogenic potential, where the cell is Flk1 positive, Sca1 positive, vimentin positive and negative for c-kit, CD34, CD45, CD31, and cytokeratin. In one embodiment, the cell is selected as Flk1 positive, Sca1 positive, vimentin positive, c-kit negative, CD34 negative, CD45 negative, CD31 negative, or cytokeratin negative using an immunoassay, such as analytical flow cytometry.

In yet another aspect, the invention features an isolated population of cells having tubulogenic potential, where at least 50%, 60%, 75%, 85%, 90%, 95% or more of the cells are Flk1 positive Sca1 positive and CD34 negative.

In yet another aspect, the invention features an isolated population of kidney cells having tubulogenic potential, where at least 50%, 60%, 75%, 85%, 90%, 95% or more of the cells are Flk1 positive Sca1 positive.

In various embodiments of the previous aspects, the population is derived from adult or embryonic kidney. In other embodiments of the previous aspects, at least 50%, 60%, 75%, 85%, 90%, 95% or more of the cells present in the population fail to express or express reduced levels of a polypeptide selected from any one or more of c-kit, CD34, CD45, CD31, and cytokeratin. In other embodiments of the previous aspects, at least 50%, 60%, 75%, 85%, 90%, 95% or more of the cells present in the population are vimentin positive.

In yet another aspect, the invention features an isolated population of adult kidney cells having tubulogenic potential, where where at least 50%, 60%, 75%, 85%, 90%, 95% or more of the cells are Flk1 positive, Sca1 positive, vimentin positive and negative for c-kit, CD34, CD45, CD31, and cytokeratin.

In another aspect, the invention features a method of identifying a cell having tubulogenic potential, the method involving the steps of identifying a cell that is Flk1 positive and Sca1 positive; and identifying the cell as failing to express or expressing reduced levels of a polypeptide selected from any one or more of c-kit, CD34, CD45, CD31, and cytokeratin, thereby identifying a cell having tubulogenic potential. In various embodiments, the identifying negative selection is performed prior to, during, or after the positive selection. In another embodiment, the method further involves the step of isolating the identified cell. In still other embodiments, the identifying is in an immunoassay (e.g., analytical flow cytometry), by cell sorting, or by by affinity selection. In one embodiment, the Flk1 Sca1 positive cells express detectable cell surface levels of Flk1 and Sca1. In another embodiment, the method further involves producing a kidney cell line from the isolated cell and expanding said cell line.

In another aspect, the invention features a method for treating or preventing a kidney disease or disorder in a subject in need thereof, the method involves administering an isolated cell or population of a previous aspect to the subject, thereby treating a kidney disease or disorder in said subject.

In yet another aspect, the invention features a method for treating or preventing a kidney disease or disorder in a subject in need thereof, the method involves administering the cell or population identified according to the method of a previous aspect to the subject, thereby treating a kidney disease or disorder in said subject.

In still another aspect, the invention features a method for regenerating a renal tubule in a subject in need thereof, the method involves administering an isolated cell or population of a previous aspect to the subject, thereby regenerating a renal tubule of said subject.

In various embodiments of the previous aspects, the administering is by direct injection to a kidney, by systemic or by local injection (e.g., via ureteric branches in the kidney). In still other embodiments, the subject has an ischemic injury, immune injury, trauma, or toxic injury.

In another aspect, the invention features a pharmaceutical composition for treating a kidney disease or disorder containing an effective amount (e.g., at least 100,000, 250,000, 500,000, 1×10⁶, 1×10⁷, or more of a cell or cell population of any previous aspect in a pharmaceutically acceptable excipient.

In yet another aspect, the invention features an kit for treatment of a kidney disease or disorder, the kit containing a cell or cell population of any previous aspect. In one embodiment, the kit further contains written instructions for using said cell for the treatment of a subject having a kidney disease or disorder. In another embodiment, the kidney disease or disorder is ischemic, toxic, traumatic or immune injury.

In various embodiments of the above methods, the method further involves the step of obtaining the cell or population of a previous aspect.

In various embodiments of any of the above aspects, fewer than about 15%, 10%, 5%, 3%, 2% or 1% of the cells express detectable levels of a polypeptide selected from any one or more of c-kit, CD34, CD45, and CD31. In various embodiments of the above aspects, the cell or population is selected as Flk1 positive, Sca1 positive, vimentin positive, c-kit negative, CD34 negative, CD45 negative, CD31 negative, or cytokeratin negative, for example, using an immunoassay, analytical flow cytometry (e.g., FACS), or affinity selection. Preferably, the cell or population is human. In still other embodiments of any previous aspect, the cell or population is capable of repairing or regenerating a renal tubule in vivo or in vitro.

The invention provides compositions and methods for the treatment of kidney disease. Other features and advantages of the invention will be apparent from the detailed description, and from the claims.

### DEFINITIONS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

By “affinity selection” is meant any selection method that depends on binding affinity. For example, the selection of a cell that selectively binds or that specifically binds to a target molecule.

By “ameliorate” is meant decrease, suppress, attenuate, diminish, arrest, or stabilize the development or progression of a disease.

The terms “comprises”, “comprising”, and are intended to have the broad meaning ascribed to them in U.S. Patent Law and can mean “includes”, “including” and the like.

By “derived” is meant that a cell or progenitor thereof was isolated from a tissue or organ where it naturally occurs.

By “disease” is meant any condition or disorder that damages or interferes with the normal function of a cell, tissue, or organ, such as a kidney.

By “exogenous” is meant a nucleic acid molecule or polypeptide that is not endogenously present in the cell. The term “exogenous” would therefore encompass any recombinant nucleic acid molecule or polypeptide expressed in a cell, such as foreign, heterologous, and over-expressed nucleic acid molecules and polypeptides.

By “kidney disease or disorder” is meant any pathology that perturbs the normal function of the kidney. Kidney diseases or disorders include acute and chronic conditions related to ischemic, immune, toxic, or traumatic injury to the kidney.

By “immunoassay” is meant an assay that employs an immunological reaction, for example, antibody binding to an antigen. Examples of immunological assays include FACs, ELISAs, Western blots, immunoprecipitations, and other assays known to the skilled artisan.

By “isolated cell” is meant a cell that is separated from the molecular and/or cellular components that naturally accompany the cell.

By “modulate” is meant positively or negatively alter. Exemplary modulations include a 1%, 2%, 5%, 10%, 25%, 50%, 75%, or 100% change.

The term “obtaining” as in “obtaining the agent” is intended to include purchasing, synthesizing or otherwise acquiring the agent (or indicated substance or material).

By “positive” is meant that a cell expresses a detectable level of a marker.

By “negative” is meant that a cell expresses an undetectable level of a marker or a reduced level of marker, such that the cell can be distinguished in a negative selection from a population of unselected cells.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “regenerate” is meant capable of contributing at least one cell to the repair or de novo construction of a tissue or organ.

The term “subject” as used herein refers to a vertebrate, preferably a mammal (e.g., dog, cat, rodent, horse, bovine, rabbit, goat), preferably a human.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

By “tubulogenic potential” is meant capable of repairing or regenerating a nephrotic tissue under appropriate in vivo or in vitro conditions. Preferably, a cell having tubulogenic potential repairs or regenerates a renal tubule.

The term “tubulogenesis” denotes the de novo construction of three-dimensional cell aggregates containing lumens within the interior of the cell clusters. Such lumens are bordered by tubule cells possessing a polarized epithelial cell phenotype with extensive microvilli formation and tight junctional complexes along the laminal border.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

### BRIEF DESCRIPTION OF THE DRAWINGS

FIGS. 1A-1C show the results of cell sorting experiments used to isolate Flk1⁺Sca1⁺ adult renal cells. FIG. 1A shows the four populations of cells identified via FACS sorting when adult renal cells were stained for Flk1 and Sca1 expression using PE- and FITC-labeled antibodies, respectively, with gating used to select cells with single and double antibody labeling. FIG. 1B shows comparable forward to side scatter relationships for cells exhibiting single and double labeling. FIG. 1C presents a table of the average cell proportions observed for different surface marker groups, wherein only approximately 0.35% of adult renal cells expressed both Flk1 and Sca1.

FIGS. 2A-2F show that Flk1⁺Sca1⁺ cells contributed to tubular structures following ischemia reperfusion injury to a significantly greater extent than control groups (Flk1⁺Sca1^{—}cells, Flk1⁻Sca1⁺ cells, Flk1⁻Sca1⁻cells, and sham control). FIG. 2A shows X-gal staining of sagittal sections obtained from approximately the same organ depth (selected from the central 100 sections) following administration of the indicated cell populations (at 100× original magnification, size bar: 100 μm), which demonstrated that increased beta-galactosidase staining was observed for Flk1⁺Sca1⁺ cells. FIG. 2B shows X-gal staining plus nuclear fast red counterstain in such sections (at 400× original magnification, size bar: 100 μm). FIG. 2C shows statistical analysis of the number of injected Flk1⁺Sca1⁺ cells that contributed to nephron structures, with cells counted at 100× magnification. FIG. 2D shows beta-galactosidase-labeled Flk-1⁺Sca1⁺ donor cells in consecutive 5μ recepient kidney sections (#92 and 93, respectively) at an original magnification of 40×. FIG. 2E shows CFSE labeling of Flk-1⁺Sca1⁺ donor cells. FIG. 2F shows colocalization of beta-galactosidase and CFSE labels in such cells.

FIGS. 3A and 3B show that Flk1⁺Sca1⁺ cells gave rise primarily to proximal tubular cells after ischemia reperfusion injury. FIG. 3A shows images for anti-beta-galactosidase immunofluorescence that demonstrated the high progenitor potential of Flk1⁺Sca1⁺ cells after ischemia reperfusion injury, comparable to the results of X-gal staining shown in FIGS. 2A-2C. From left to right, the panels of FIG. 3A present immunofluorescence results for a Rosa26 positive control, a wild-type negative control, a wild-type recipient kidney injected with Flk1⁺Sca1⁺ cells, and a normal saline-injected sham negative control of wild-type mouse, with all original magnifications at 400× and the size bar indicating 100 μm. FIG. 3B shows images obtained for colocalization studies of anti-beta-galactosidase with the tubular markers megalin, aquaporin 1 and 2, and THP. New tubules were positive for megalin and aquaporin1, demonstrating their proximal tubular specialization, with all images possessing original magnifications of 200× and the size bar indicating 100 μm.

FIGS. 4A-4E show that Flk1⁺Sca1⁺ cells gave rise to tubular epithelium in cultured metanephroi and contributed to a higher degree to tubule formation than cells not expressing both surface markers. FIG. 4A shows images of beta-galactose staining in, from left to right, positive whole-mount staining in a Rosa26 mouse; positive section staining in a Rosa26 mouse; negative section staining in a wild-type mouse; and the right hand panel shows the locations of metanephroi injections, with stars indicating injection sites for sorted cells in E13.5 cultured metanephroi (original magnification: 80×; size bar: 100 μm). FIG. 4B shows whole-mount X-gal staining for metanephroi injected with cells from all experimental groups (original magnification: 80×). FIG. 4C shows an overview of X-gal-stained sections with nuclear fast red counterstain. FIG. 4D shows magnifications of the sections indicated in FIG. 4C, with the left hand panel showing a complete tubular cross-section of newly formed tubule in the insert of this image (original magnification: 200×. Size bar: 100 μm). FIG. 4E shows a histogram presenting the results of statistical analysis of the contribution of injected cells to tubules, wherein only tubules exhibiting three or more X-gal positive cells were counted for comparisons between different populations of sorted cells.

FIG. 5 shows that Flk1⁺Sca1⁺ adult renal cells exhibit characteristics of mesenchymal cells, including a high degree of vimentin (top row of image panels), but not cytokeratin (bottom row of image panels), expression. Left panels show a control image of marker immunofluorescence in the adult renal cortex, while middle images show marker immunofluorescence of sorted Flk1⁻ and Sca1⁻ expressing cells. A histogram at right shows the percentage of vimentin-positive cells in the sorted cell populations.

FIGS. 6A and 6B show that Flk1⁺Sca1⁺ population cells retained the capacity to give rise to tubular structures after ischemia reperfusion injury when cells expressing certain hematopoietic and mesenchymal stem cell markers were excluded. FIG. 6A shows FACS used to deplete cells that expressed CD34, CD45, and c-kit markers from the injected population of cells. FIG. 6B shows representative sections stained for beta-galactosidase and a nuclear fast red counterstain, demonstrating that cells derived from sorted and injected Flk1⁺Sca1⁺c-kit⁻CD34⁻CD45⁻ localized to tubular structures.

FIG. 7 shows the physiological localization of Flk1⁺Sca1⁺ cells to the peritubular cortical region in adult kidneys. The top series of panels shows control images of Flk1 and Sca1 immunofluorescence labeling, including negative controls without primary antibody incubation, while the bottom panels show images of Flk1 and Sca1 colocalization by immunofluorescence in the cortex region of the adult kidney. Flk1⁺Sca1⁺ cells were situated in the peritubular interstitium.

### DETAILED DESCRIPTION OF THE INVENTION

As described in more detail below, the present invention provides cellular compositions for the treatment or prevention of kidney disease. The invention is based, at least in part, on the discovery of a population of cortical peritubular Flk1- and Sca1-expressing kidney cells having a high tubulogenic potential both after ischemia reperfusion injury and during embryonic kidney culture. These Flk1+Sca1+ kidney cells constitute an important progenitor population with relevance for tubular epithelial maintenance and repair. Preferably, these cells also express the mesenchymal marker vimentin. In other preferred embodiments, cells expressing Flk1+Sca1+ fail to express detectable levels or express reduced levels of any one or more of CD34, CD45, and c-kit.

Isolation of Flk-1 Sca-1 Expressing Cells

The unpurified source of cells for use in the methods of the invention may be any tissue or organ known in the art. In preferred embodiments, the tissue or organ used is an adult or embryonic kidney. Preferably, cells of the invention are derived from the peritubular cortex of the kidney (e.g., an adult or embryonic kidney) and have tubulogenic potential.

Various techniques can be employed to separate or enrich for the desired cells. Such methods include a positive selection for cells expressing any one or more of Flk-1, Sca1 and vimentin. If desired, a negative selection is carried out for the isolation of cells that do not express at detectable levels any one or more of CD34, CD45, and c-kit. In one embodiment, cells selected for use in the methods of the invention express Flk-1, Sca1 and vimentin, and express virtually undetectable or reduced amounts of CD34, CD45, and c-kit relative to an unselected population of cells isolated from the kidney. mAbs are particularly useful for identifying markers associated with the desired cells and are useful for both positive and negative selections.

If desired, magnetic bead separations can be used initially to remove large numbers of irrelevant cells (i.e., cells expressing CD34, CD45, and c-kit). Given that Flk1+Sca1+ cells made up ˜0.3% of cells isolated from adult kidney, preferably, at least about 80%, usually at least 70% of the total kidney cells will be removed prior to isolation of the desired cell type.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g. plate, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). Preferably, the cells are collected in a medium comprising fetal calf serum (FCS) or bovine serum albumin (BSA) or any other suitable, preferably sterile, isotonic medium.

Selected cells of the invention may be employed in therapeutic or prophylactic methods following isolation. Accordingly, the present invention provides methods of treating kidney disease and/or disorders or symptoms thereof which comprise administering a therapeutically effective amount of a pharmaceutical composition comprising a cell identified according to the methods described herein to a subject (e.g., a mammal such as a human). Thus, one embodiment is a method of treating a subject suffering from or susceptible to a kidney disease or disorder or symptom thereof. The method includes the step of administering to the mammal a therapeutic amount of an amount of a cell herein sufficient to treat the disease or disorder or symptom thereof, under conditions such that the disease or disorder is treated.

The methods herein include administering to the subject (including a subject identified as in need of such treatment) an effective amount of a cellular composition described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of a cellular composition described herein, such as a cell isolated from the adult kidney herein to a subject (e.g., animal, human) in need thereof, including a mammal, particularly a human. Such treatment will be suitably administered to subjects, particularly humans, suffering from, having, susceptible to, or at risk for a kidney disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which ischemic damage or kidney toxicity may be implicated.

In one embodiment, the invention provides a method of monitoring treatment progress in connection with a kidney disease. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with kidney disease, in which the subject has been administered a therapeutic amount of a cellular composition described herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

In some embodiments, it may be desirable to maintain the selected cells in culture for hours, days, or even weeks prior to administering them to a subject. Media and reagents for tissue culture are well known in the art (see, for example, Pollard, J. W. and Walker, J. M. (1997) Basic Cell Culture Protocols, Second Edition, Humana Press, Totowa, N.J.; Freshney, R.I. (2000) Culture of Animal Cells, Fourth Edition, Wiley-Liss, Hoboken, N.J.). Examples of suitable media for incubating/transporting kidney stem cell samples include, but are not limited to, Dulbecco's Modified Eagle Medium (DMEM), RPMI media, Hanks' Balanced Salt Solution (HBSS) phosphate buffered saline (PBS), and L-15 medium. Examples of appropriate media for culturing cells of the invention include, but are not limited to, Dulbecco's Modified Eagle Medium (DMEM), DMEM-F12, RPMI media, EpiLlfe medium, and Medium 171. The media may be supplemented with fetal calf serum (FCS) or fetal bovine serum (FBS) as well as antibiotics, growth factors, amino acids, inhibitors or the like, which is well within the general knowledge of the skilled artisan.

Formulations

Compositions of the invention comprising purified cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified immunoresponsive cells utilized in practicing the present invention in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the genetically modified immunoresponsive cells or their progenitors.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the genetically modified immunoresponsive cells as described in the present invention. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of genetically modified immunoresponsive cells of the invention is the quantity of cells necessary to achieve an optimal effect. The quantity of cells to be administered will vary for the subject being treated.

In a one embodiment, between 10⁴ to 10⁸, between 10⁵ to 10⁷, or between 10⁶ and 10⁷ genetically modified immunoresponsive cells of the invention are administered to a human subject. In preferred embodiments, at least about 1×10^{7,} 2×10⁷, 3×10⁷, 4×10⁷, and 5×10⁷ genetically modified immunoresponsive cells of the invention are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods of the invention.

Typically, any additives (in addition to the active stem cell(s) and/or agent(s)) are present in an amount of 0.001 to 50% (weight) solution in phosphate buffered saline, and the active ingredient is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %. Of course, for any composition to be administered to an animal or human, and for any particular method of administration, it is preferred to determine therefore: toxicity, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

Administration of Cells

Compositions comprising a selected cell of the invention (e.g., a kidney cell, or progenitor thereof, that expresses Flk-1, Sca1 and vimentin, and that fails to express CD34, CD45, and c-kit) or their progenitors can be provided systemically or directly to a subject for the treatment or prevention of a chronic or acute kidney disease or disorder, such as ischemic or toxic injury. In one embodiment, cells of the invention are directly injected into an organ of interest (e.g., a kidney). Alternatively, compositions comprising selected cells of the invention are provided indirectly to the organ of interest, for example, by administration into the circulatory system (e.g., the kidney vasculature). Expansion and differentiation agents can be provided prior to, during or after administration of the cells to increase production of cells having tubulogenic potential in vitro or in vivo.

The cells can be administered in any physiologically acceptable vehicle, normally intravascularly, although they may also be introduced into other convenient site where the cells may find an appropriate site for regeneration and differentiation. In one approach, at least 100,000, 250,000, or 500,000 cells is injected. In other embodiments, 750,000, or 1,000,000 cells is injected. In other embodiments, at least about 1×10⁵ cells will be administered, 1×10⁶, 1×10⁷, or even as many as 1×10⁸ to 1×10¹⁰, or more Selected cells of the invention can comprise a purified population of cells that expresses Flk-1, Sca1 and vimentin. Preferably, the cells fail to express (or express greatly reduced or virtually undetectable amounts) of CD34, CD45, and c-kit. Those skilled in the art can readily determine the percentage of cells in a population using various well-known methods, such as fluorescence activated cell sorting (FACS). Preferable ranges of purity in populations comprising selected cells are about 50 to about 55%, about 55 to about 60%, and about 65 to about 70%. More preferably the purity is at least about 70%, 75%, or 80% pure, more preferably at least about 85%, 90%, or 95% pure. In some embodiments, the population is at least about 95% to about 100% selected cells. Dosages can be readily adjusted by those skilled in the art (e.g., a decrease in purity may require an increase in dosage). The cells can be introduced by injection, catheter, or the like.

Compositions of the invention include pharmaceutical compositions comprising genetically modified immunoresponsive cells or their progenitors and a pharmaceutically acceptable carrier. Administration can be autologous or heterologous. For example, immunoresponsive cells, or progenitors can be obtained from one subject, and administered to the same subject or a different, compatible subject.

Selected cells of the invention or their progeny (e.g., in vivo, ex vivo or in vitro derived) can be administered via localized injection, including catheter administration, systemic injection, localized injection, intravenous injection, or parenteral administration. When administering a therapeutic composition of the present invention (e.g., a pharmaceutical composition containing a selected cell), it will generally be formulated in a unit dosage injectable form (solution, suspension, emulsion).

Accordingly, the invention also relates to a method of treating a subject having a kidney disorder. This method comprises administering to the subject an effective amount either of a stem/progenitor cell isolated as explained herein or of a cellular extract derived from such a cell.

The kidney disease or disorder to be treated can be a congenital or an acquired kidney deficiency. Examples of such diseases or disorders include, but are not limited to, ischemic injury, toxic injury, and immune injury.

In another pharmaceutical use, stem/progenitor cells of the present invention can be used genetically modified prior to their administration to a subject. For this purpose, the cells can be transformed with a nucleic acid encoding the protein that is to be produced in the cells. The nucleic acid can be introduced into a cells of the invention using any of the various methods that are well known to the skilled person, for example, using a viral vector and/or a lipid containing transfection composition such as IBAfect (IBA GmbH, Gobttingen, Germany), Fugene (Roche), GenePorter (Gene Therapy Systems), Lipofectamine (Invitrogen), Superfect (Qiagen), Metafecten (Biontex) or those ones described in the PCT application WO 01/015755). In a related embodiment, the cells of the invention, after being transformed with a nucleic acid encoding a polypeptide of choice, can be used of recombinantly producing this polypeptide.

In a further embodiment and in line with the above disclosure, the kidney cells of the invention may be genetically modified to produce a therapeutic polypeptide. Examples of such therapeutic polypeptide include, but are not limited to, a protein such as a cytokine, a growth factor such as insulin-like growth factor (IGF), epidermal growth factor (EGF), transforming growth factor beta (TGF-beta), Activin A, a bone morphogenetic protein (BMP), PDGF or a hormone as insulin or erythropoietin or a transporter protein such transferrin, a peptide such a growth factor or hormone (e.g. luteinic hormone (LSH), follicle stimulating hormone (FSH)), a small organic molecule such as a steroid hormone, an oligo- or polysaccharide, for example, heparin or heparan sulfate (cf., example WO 96/23003, or WO 96/02259 in this regard), a proteoglycan, a glycoprotein such as collagen or laminin, or a lipid, to name only a few.

Vectors

Genetic modification of selected cells of the invention can be accomplished by transforming or transducing a selected cell or population of cells comprising a desired cell type with a recombinant DNA construct. Virtually any vector or delivery system known in the art may be used to modify a cell of the invention (e.g., a kidney cell or progenitor thereof). Preferably, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the expression of a protein in cell. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can then be grown under conditions similar to those for unmodified cells, whereby the modified cells can be expanded and used for a variety of purposes. Cultivation of the kidney stem/progenitor cells of the instant invention can be carried out in any media that is suitable for cultivation of mammalian cells. Examples include conventional and commercially available media such as, but not limited to, KGM.RTM.-Keratinocyte Medium (Cambrex), MEGM-Mammary Epithelial Cell Medium (Cambrex) EpiLife medium (Cascade Biologics), Medium 171 (Cascade Biologics), DMEM, DMEM-F12 or RPMI media. Cultivation is typically carried out at conditions (temperature, atmosphere) that are normally used for cultivation of cells of the species of which the cells are derived, for example, at 37° C. in air atmosphere with 5% CO₂. In one embodiment, the cultivation is carried out using serum free, in particular bovine serum free media. The cultivation (in one passage) is performed for any suitable time the cells need for growth, typically, but by no means limited to, for about 1 to several days, for example to about 7 or about 8 days.

Methods of Treatment

Provided herein are methods for treating or preventing acute or chronic kidney disease in a subject. In particular embodiments, the invention provides methods for treating or preventing acute or chronic kidney disease related to ischemic, immune, toxic, or traumatic injury. Exemplary kidney diseases and disorders amenable to treatment using a method of the invention include, but are not limited to, Alport Syndrome, amyloidosis and kidney disease, chronic kidney disease, kidney failure, glomerular disease, glomerulonephritis, goodpasture's syndrome, iga nephropathy, interstitial nephritis, lupus nephritis, medullary sponge kidney, multicystic kidney dysplasia, nephrotic syndrome, polycystic kidney disease, renal fusion, renal tubular acidosis, renovascular conditions, simple kidney cysts, solitary kidney, tubular and cystic kidney disorders. Patients having a kidney disease or disorder are generally identified by a reduction in kidney function. Methods for assaying kidney function are known in the art and include tests to identify increased levels protein in the urine, a condition called proteinuria. (Healthy kidneys allow less than about 1 gram of protein into the urine). Increased levels of creatinine in the blood, which typically result from a decreased excretion of creatinine in the urine. A normal value for blood creatinine is 0.8 to 1.4 mg/dl. BUN (blood urea nitrogen) is a test that measures the amount of urea nitrogen (a breakdown product of protein metabolism) in the blood. A normal BUN is between 7-20 mg/dl. Higher-than-normal levels (i.e., about 10%, 25%, 50%, 75% or even as much as 2, 3, or 4-fold higher) levels in any one or more of these parameters generally indicate the presence of a kidney disease or disorder.

In general, the methods comprise administering a selected cell of the invention in an amount effective to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the antigen-binding fragment administered.

Suitable human subjects for therapy typically comprise two treatment groups that can be distinguished by clinical criteria. The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Kits

The invention provides kits for the treatment or prevention of kidney disease, particularly kidney disease related to an ischemic or toxic injury. In one embodiment, the kit includes a therapeutic or prophylactic composition containing an effective amount of a cell isolated using the methods described herein (e.g., a kidney cell that expresses vimentin, VEGF receptor −2 (Flk-1) and stem cell antigen-1 (SCA-1), but that fails to express, or expresses only limited amounts of hematopoietic markers CD34, CD45, and c-KIT) in unit dosage form. In some embodiments, the kit comprises a sterile container which contains a therapeutic or prophylactic cellular composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired a cell of the invention is provided together with instructions for administering the agent to a subject having or at risk of developing kidney disease, such as kidney ischemic or toxic injury. The instructions will generally include information about the use of the composition for the treatment or prevention of kidney disease. In other embodiments, the instructions include at least one of the following: description of the therapeutic agent; dosage schedule and administration for treatment or prevention of ischemia or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

Exemplification

The following examples are put forth so as to provide those of ordinary skill in the art with a complete disclosure and description of how to make and use the assay, screening, and therapeutic methods of the invention, and are not intended to limit the scope of what the inventors regard as their invention.

The mammalian adult kidney possesses the inherent potential for tubular regeneration and recovery following ischemic, immune or toxic injury. The following examples describe the identification of an intrarenal cell population isolated from adult mouse kidney constituting ˜0.2% of the total extracted renal cells, which were shown to possess the potential to differentiate into renal tubular epithelium in vivo following an acute ischemic insult, and in vitro in cultured embryonic metanephroi. This vimentin⁺ cytokeratin⁻ cell population was identified to express both the vascular-endothelial growth factor receptor-2 (Flk-1) and the stem cell antigen-1 (SCA-1), but did not express the hematopoietic markers CD34, CD45, and c-KIT. Flk-1⁺SCA-1⁺ cells were found mainly in renal interstitium and were significantly increased following acute renal reperfusion injury.

### Example 1

### Isolation of a Flk-1⁺Sca1⁺ Cell Population From Adult Mouse Kidney

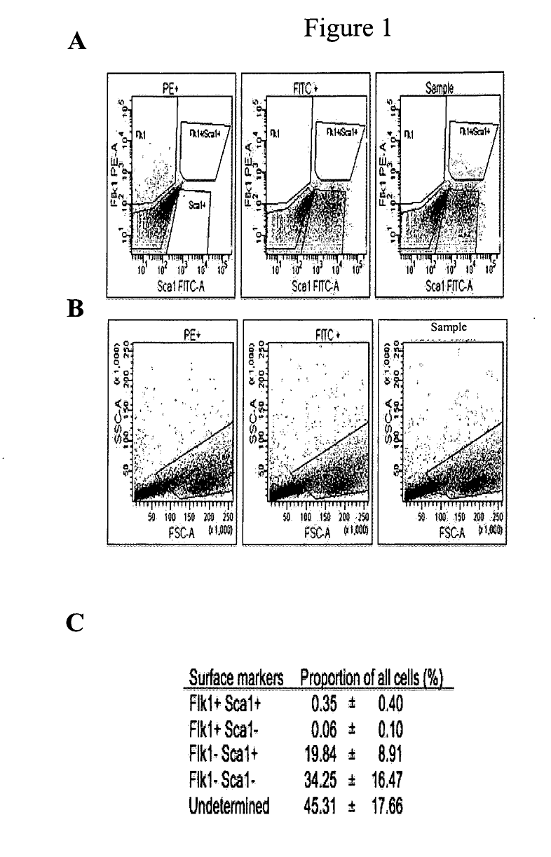

A renal Flk1⁺ and Sca1-expressing (Flk1⁺Sca1⁺) cell population was obtained from single cell suspensions of adult murine kidneys by fluorescence activated cell sorting (FACS; refer to FIGS. 1A and 1B). For each experiment, matched unlabeled (data not shown) and single labeled control tubes (refer to FIGS. 1A and 1B, left panels) were prepared for proper gating of single and double positive signals. While the proportion of Flk1⁻Sca1⁺ cells was relatively high at approximately one-fifth of cells examined (refer to FIGS. 1A and 1C), Flk1⁺Sca1⁺ cells made up less than 0.5% of the total cell population (n≧500,000 cells examined). Size and internal complexity of these cells did not differ from other cells, as assessed by FACS forward and side scatter (refer to FIG. 1B).

### Example 2

### Flk-1⁺Sca1⁺ Cells Exhibited Tubulogenic Potential Upon Transplant to Ischemic Adult Mouse Kidney

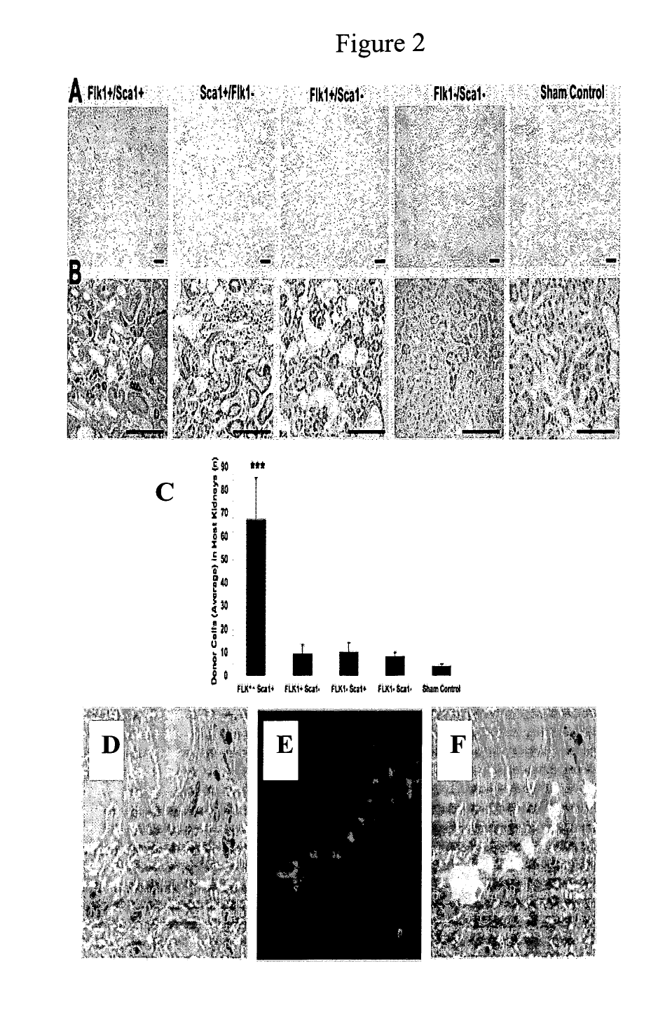

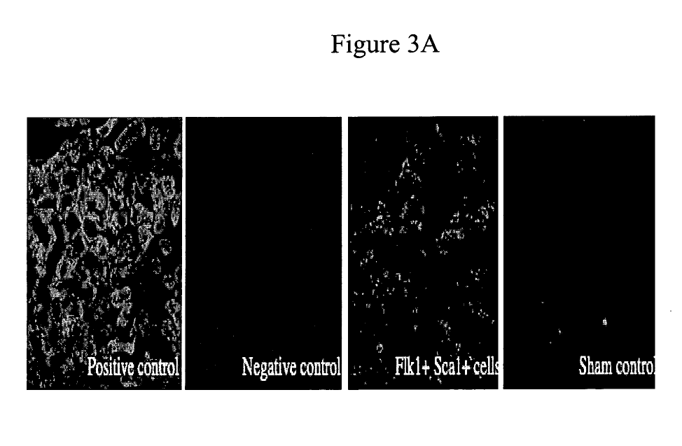

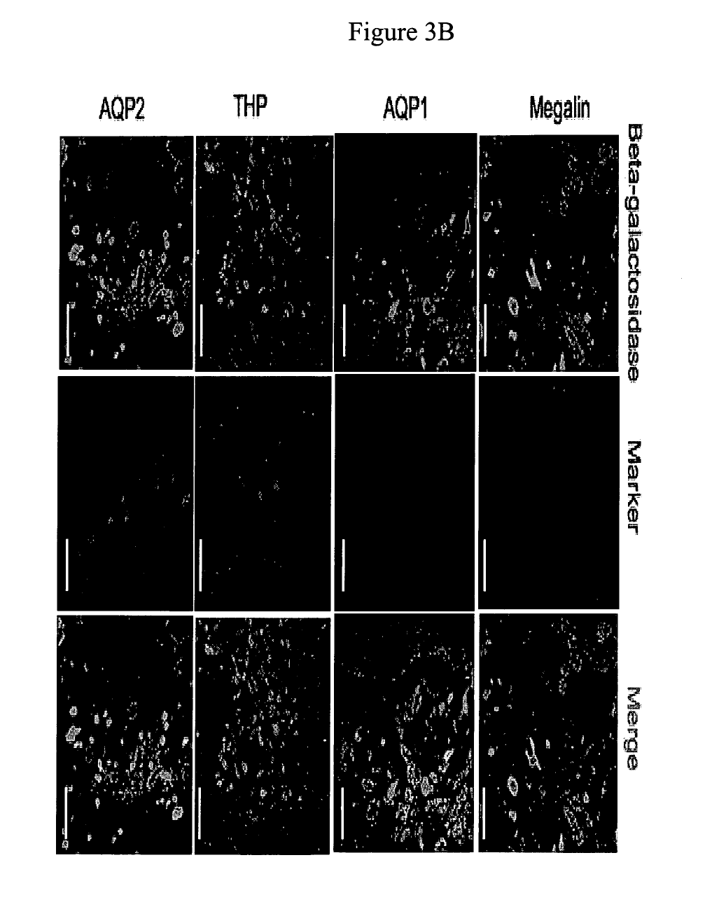

In order to test the tubular regenerative potential of Flk1⁺Sca1⁺ cells, the contribution of injected Flk1⁺Sca1⁺ cells to host tissue was tracked by assessment of bacterial beta-galactosidase expression (Flk1⁺Sca1⁺ cells were obtained from the kidneys of male Rosa26/LacZ⁺ mice that expressed bacterial beta-galactosidase (β-gal) in all tissues from a knock-in LacZ gene (Friedrich and Soriano (1991) Genes Dev. 5: 1513-23)). Beta-galactosidase expression was detected using chromogenic substrates (refer to FIG. 2) or via antibody labeling (refer to FIG. 3). Sorted cells were injected into female C57BL/6 host mice after those animals were exposed to ischemia reperfusion injury by renal pedicle clamping for 26 minutes (refer to Example 9 below, and to Kale et al. (2003) J Clin Invest. 112: 42-9; and Kelly et al. (1996) J Clin Invest. 97: 1056-1063). Twelve days later, the reconstitution of tubules and differentiation of transplanted cells in recipient kidneys were checked in serial cryostat sections by beta-galactosidase staining.

Flk1⁺Sca1⁺ cells contributed best to renal tubules in comparison to control groups. Bacterial beta-galactosidase was detected to a significant degree in Flk1⁺Sca1⁺ cell-injected chimeras following ischemia reperfusion injury (n≧5; refer to FIGS. 2A and 2B). In other chimeras, such as those injected with Flk1⁻Sca1⁺ cells, Flk1⁺Sca1⁻ cells, Flk1⁻Sca1⁺ cells, and in the normal saline-injected sham control group, only a small number of cells stained for beta-galactosidase expression. Most of the beta-galactosidase positive cells from Flk1⁺Sca1⁺ cell recipient mice formed structures resembling kidney tubules, assessed morphologically (refer to FIGS. 2A and 2B). The beta-galactosidase positive cells from other chimeric groups were only occasionally observed to be in a pattern of scattered distribution.

Quantitative studies for different chimeras were performed on the central 100 serial sections, and the average number of beta-galactosidase positive cells integrated into tubules per section was calculated. Single cells in other localizations were not counted. Positive staining was significantly higher in Flk1⁺Sca1⁺ cell-recipient mice than in any other group (P=0.005; FIG. 2C).

An additional demonstration of the donor origin of transplanted cells was achieved using the fluorochrome, carboxyfluoroscein succinimidyl ester (CFSE) to stain Rosa26/LacZ⁺-derived Flk1⁺Sca1⁺ cells. Such cells were labeled with CFSE prior to transplantation, and consecutive 5 μm sections of chimeric kidneys were stained with beta-galactosidase and assessed for immunofluorescence (refer to FIGS. 2D and 2E, respectively). Beta-galactosidase and CFSE were found to colocalize in many tubules (refer to FIG. 2F).

Results similar to those for beta-galactosidase detection by chromogenic substrate development (refer to FIG. 2) were obtained via antibody labeling (refer to FIG. 3A). Kidneys from Rosa26 control mice showed generalized staining for beta-galactosidase (positive control; refer to FIG. 3A), while host C57BL/6 mice did not exhibit any signal (negative control; refer to FIG. 3A), indicating that the beta-galactosidase immunostaining was specific. The kidneys of nontransgenic mice that were injected with Rosa26 mouse-derived Flk1⁺Sca1⁺ cells showed areas containing beta-galactosidase-positive cells in a tubule-resembling configuration (Flk1⁺Sca1⁺ cells; refer to FIG. 3A). Compared with the staining in the Rosa26 mouse kidney, a heterogeneous pattern of beta-galactosidase staining was seen within the same tubule, indicating that some of the tubular cells were derived from Flk1⁺Sca1⁺ of Rosa26 mice during regeneration. In contrast, in sham control kidneys from mice that did not receive Flk1⁺Sca1⁺ cell transplants, staining was negative (sham control; refer to FIG. 3A).

### Example 3

### Flk1⁺Sca1⁺ Cells Primarily Differentiated into Proximal Tubular Epithelial Cells Following Ischemia Reperfusion Injury

To identify specific nephron segments that were derived from Flk1⁺Sca1⁺ cells, a number of renal tubule markers were examined in injured kidneys for colocalization with beta-galactosidase-expressing Flk1⁺Sca1⁺ cells. Kidney sections were stained with antibodies to beta-galactosidase and to markers of specific nephron segments within the same sections and evaluated for colocalization by fluorescence microscopy (refer to FIG. 3B). Megalin, a specific marker of the renal proximal tubule, was located in the brush border of the renal proximal tubule (refer to FIG. 3B, top row of panels). The merged image demonstrated that most of the beta-galactosidase-positive cells also expressed megalin. Similarly, costaining with an antibody to aquaporin 1 (AQP1), a proximal tubule-specific brush border glycoprotein, showed AQP1 expression in beta-galactosidase-positive tubular cells (refer to FIG. 3B, second row of panels from top). About 90% of the proximal tubules contained cells that costained with antibodies to beta-galactosidase and to AQP1. These results demonstrated that, during kidney regeneration, some of the renal cells that were derived from beta-galactosidase-expressing Flk1⁺Sca1⁺ cells adopted a renal proximal tubular cell phenotype.

Cells of the distal nephron were also examined for the expression of beta-galactosidase. There was no colocalization of beta-galactosidase and Tamm-Horsfall protein (THP; refer to FIG. 3B, third row of panels from top), a marker expressed in the thick ascending limb of the loop of Henle (TALH) and in the distal tubules. Likewise, there was lack of colocalization between beta-galactosidase and NKCC2, a sodium-potassium-chloride co-transporter expressed in the TALH (data not shown). Beta-galactosidase also was not expressed in collecting ducts that stained positive for aquaporin 2 (AQP2; refer to FIG. 3B, bottom row of panels).

These results demonstrated that, during kidney regeneration, some of the renal cells that were derived from beta-galactosidase-expressing Flk1⁺Sca1⁺ cells had adopted a renal proximal tubular cell phenotype.

### Example 4

### Transplanted Flk1⁺Sca1⁺ Cells Developed into Tubules During Metanephros Culture

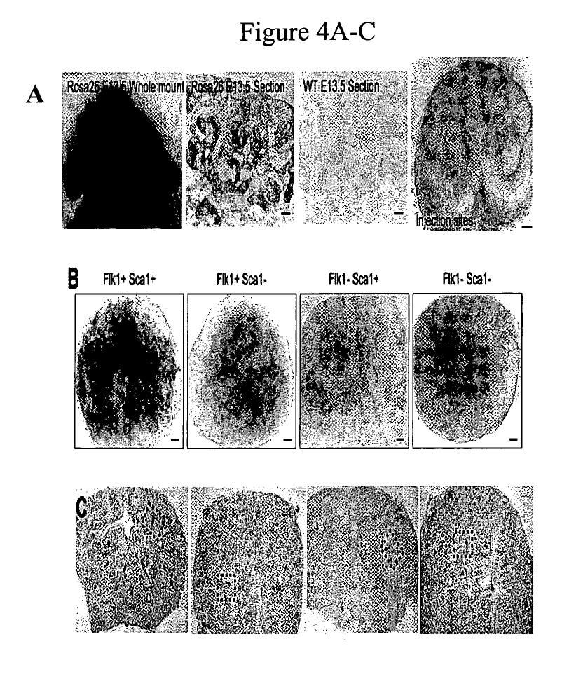

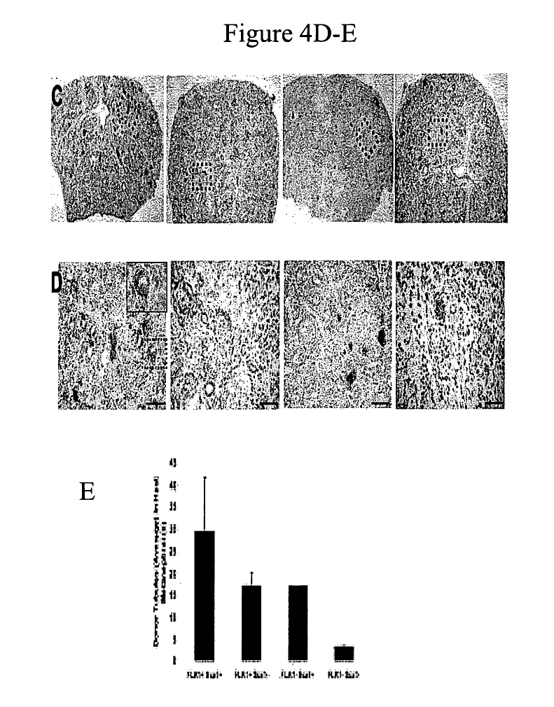

The ability of murine adult renal Flk1⁺Sca1⁺ cells to serve as potent progenitor cells specifically for tubule development was further investigated via transplantation of such cells into cultured embryonic metanephroi. Control experiments performed upon whole mount samples and tissue sections of Rosa26 metanephroi showed significant X-gal staining, indicative of robust beta-galactosidase expression, whereas wild-type embryonic kidneys showed no such staining (refer to FIG. 4A, left panels). Freshly sorted cells were microinjected into cultured metanephroi of C57BL/6 mice obtained at embryonic day 12.5 (E12.5) after timed matings. Cells were injected in close proximity to ureteric branches, both at the organ hilum and, mainly, in the periphery (refer to FIG. 4A, right panel).

When the microinjected kidneys were processed for beta-galactosidase staining after seven days of organ culture, substantial beta-galactosidase staining was observed in whole mounts for Flk1⁺Sca1⁺ cell-injected metanephroi (refer to FIG. 4B, left panel). In comparison, staining in metanephroi injected with cells of the other groups that lacked observable simultaneous expression levels of those two markers was much less (n≧8; refer to FIG. 4B). Upon tissue sectioning, the staining was identified primarily in cells contained in tubule-like configurations (refer to FIGS. 4C and 4D). Again, Flk1⁺Sca1⁺ cell-injected metanephroi demonstrated more positive tubules than the other cell populations, such as Flk1⁺Sca1⁻, Flk1⁻Sca1⁺, and Flk1⁻Sca1⁻ cells. Quantitative studies among the different cell populations was carried out by determining the average number of intact tubules having three or more positive cells counted in all sections, ignoring individual surviving cells. This quantification further demonstrated a significant advantage in the potential of Flk1⁺Sca1⁺ cells to contribute to tubule formation (refer to FIG. 4E), as was shown above via qualitative staining assessments.

### Example 5

### Flk1⁺Sca1⁺ Cells Exhibited Characteristics of Mesenchymal Cells and were Localized Within the Peritubular Cortical Region in Adult Renal Kidneys

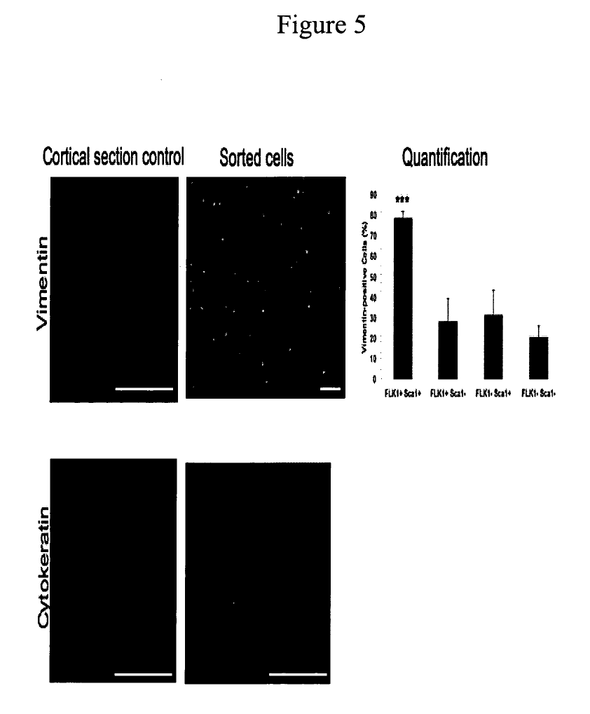

After establishing that Flk1⁺Sca1⁺ cells possessed the ability to generate new tubules significantly more effectively than control cell populations, the characteristics of these cells were further assessed ex vivo. To evaluate the tissue of origin for Flk1⁺Sca1⁺ cells, sorted Flk1⁺Sca1⁺ cells and control groups were processed for vimentin (a mesenchymal marker) and cytokeratin (an epithelial marker) immunofluorescence. Cells were transiently cultured for 4 hours in poly-L-lysine pretreated chamber slides in order to allow attachment, or they were subjected to cytospin centrifugation to harvest cells on slides. 77% of Flk1⁺Sca1⁺ cells were identified as vimentin-positive, whereas less than 2% of total cells were cytokeratin-positive (refer to FIG. 5). Accordingly, the Flk1⁺Sca1⁺ cell population consisted primarily of mesenchymal, not epithelial, cells.

### Example 6

### Flk1⁺Sca1⁺ Cells Did Not Express Additional Stem Cell Markers at Significant Levels

Flk1⁺Sca1⁺ cells expressed other markers of hematopoietic, mesenchymal, and endothelial stem cells to only a minor degree:

TABLE 1
Expression of Additional Stem Cell Markers in
Flk1⁺Sca1⁺ Cells

| | Proportion of |
|---|---|
| Stem Cell Marker | Flk1⁺Sca1⁺ Cells (%) |
| c-kit | 3.45 ± 1.61 |
| CD34 | 3.70 ± 2.95 |
| CD45 | 5.90 ± 5.09 |
| CD31 | 12.80 ± 0.00 |

The expression of c-kit, CD34, and CD45 at or in less than 5% of Flk1⁺Sca1⁺ cells was unlikely to represent circulating hematopoietic stem cells lodged in the kidney, as careful kidney perfusion at harvest was performed. While the expression of c-kit and CD45 was observed to demonstrate a slight overlap in signals, no such overlap was observed for the other markers presented in Table 1.

### Example 7

### Flk1⁺Sca1⁺ Cells Retained Tubulogenesis Potential After Exclusion of a Hematopoietic Stem Cell-Resembling Subtype of Cells

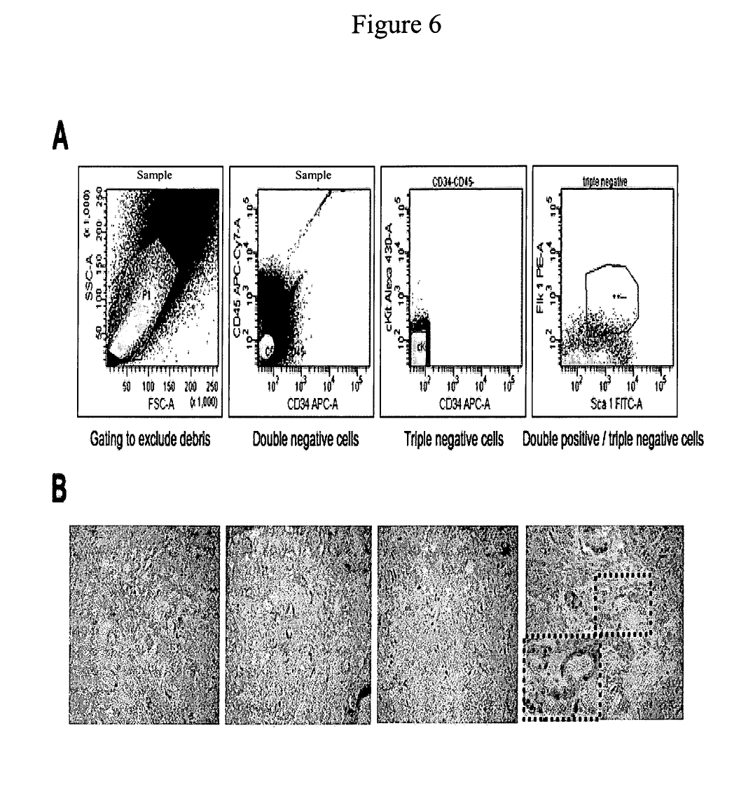

Further confirmation that the Flk1⁺Sca1⁺ cells isolated from adult kidney cells were distinct from previously identified stem cell populations was obtained by assessing Flk1⁺Sca1⁺ cells for tubulogenesis capability following the exclusion of a hematopoietic stem cell-resembling subtype of cells (refer to FIG. 6A). To exclude such hematopoietic stem cell-like cells, the suspension of all murine adult renal cells was initially depleted of c-kit, CD34, and CD45, and Flk1- and Sca1-expressing cells were then collected. Injection of this isolated population of Flk1⁺Sca1⁺c-kit⁻CD34⁻CD45⁻ cells after ischemia reperfusion injury was performed according to the same protocol used in the Examples above for Flk1⁺Sca1⁺ cell populations, and demonstrated that Flk1⁺Sca1⁺c-kit⁻CD34⁻CD45⁻ cells were still able to give rise to tubular structures (refer to FIG. 6B).

### Example 8

### Flk1⁺Sca1⁺ Cells were Physiologically Localized in Murine Cortical Interstitium Between Tubules

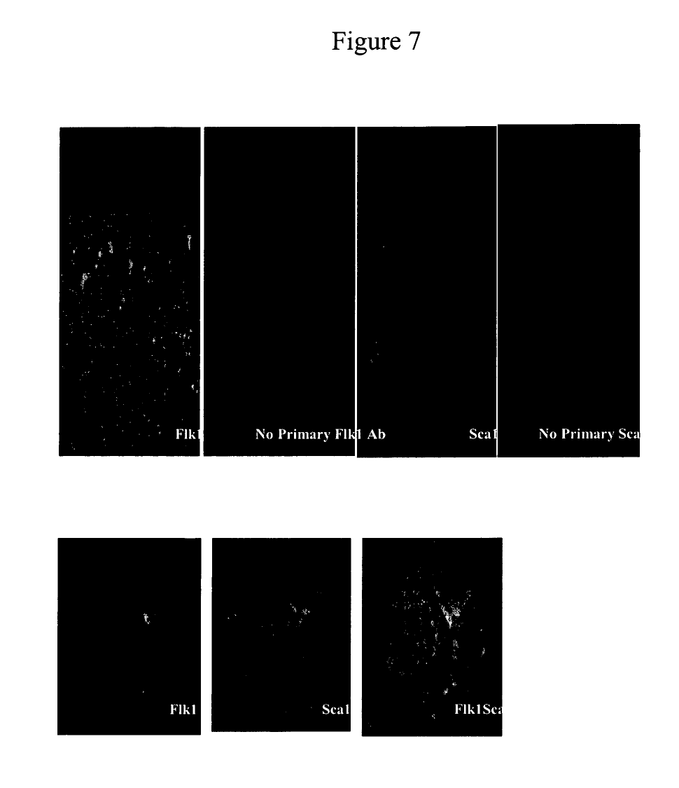

Having identified the mesenchymal character of the Flk1⁺Sca1⁺ cell population, the physiologic localization of such cells in the murine adult kidney was examined. Using double immunofluorescence, Flk1⁺Sca1⁺ cells were demonstrated in the normal kidney parenchyma, in the interstitial tissue between cortical tubules (refer to FIG. 7). Control experiments performed on murine kidney cortex showed that Flk1 and Sca1 were specifically labeled by immunofluorescence (refer to FIG. 7A), and colocalization assessment revealed double staining for both markers in cells situated in the peritubular cortical compartment (refer to FIG. 7B). Thus, the very small portion of kidney cells identified as Flk1⁺Sca1⁺ cells were not only of mesenchymal origin but also were physiologically localized in the murine cortical interstitium between tubules.

The results describe above were obtained using the following methods and materials.

Isolation of Adult Renal Flk1⁺Sca1⁺ Cell Population

C57BL/6 and Rosa26 mice were purchased from the Jackson Laboratory and were housed in the animal research facilities of Massachusetts General Hospital. All animal experiments were performed in compliance with institutional review board requirements. Kidney cells were harvested from Rosa26 mice at 6 to 8 weeks of age, and the candidate cell population was isolated based on positive or negative selection with fluorescence-activated cell sorting (FACS). Briefly, mice were anesthetized, the right atrium was opened, and mice were perfused with 15 ml of suitable cell culture media (Dulbecco's modified eagle medium (DMEM), purchased from Invitrogen) injected into the left ventricle. Efficient perfusion was judged by observing the liver of the perfused mouse change color from red to pale. Kidneys were harvested, kidney capsules were removed, and kidneys were minced to pieces on a sterilized glass dish. Incubation with collagenase II (4 mg/ml in cell culture media; Worthington) at 37° C. for 1 hour with occasional agitation was followed by washing with 10% fetal calf serum (FCS) in cell culture media. Cell suspensions were filtered through 50 μm and 30 μm filters (BD Science) on ice, and single cells were resuspended in 1% FCS in cell culture media at a concentration of 1 million cells per 100 μl. Fc receptors were blocked with mouse CD16/32 antibodies (BD Pharmingen). For direct labeling, cells were incubated with fluorophore-conjugated antibodies (PE rat anti-mouse Flk1, FITC rat anti-mouse Sca-1, APC rat anti-mouse CD34, APC-Cy7 rat anti-mouse CD45, Alexa 430 rat anti-mouse c-kit; all BD Biosciences) or control IgG (PE or FITC rat IgG-2a, kappa isotype control, BD Biosciences) before sorting by FACS (FACSAria or FACSVantage Cell Sorter, BD). After 30 minutes of antibody incubation on ice, cells were washed twice with 10% FCS in cell culture media, prior to sorting. For analytical flow cytometry, cells were labeled with APC rat anti-mouse CD31 antibody (BD Biosciences) in some experiments. Light protection was performed for each step in which fluorescent antibodies were involved by using light-protective tubes (Eppendorf tubes; Denville Scientific) or by wrapping tubes with foil. Enhanced selection of single cells, as opposed to aggregates, during FACS was achieved by gating on higher height values in comparison to area measurements in the forward scatter plots. All liquid reagents used for cell isolation and tissue culture were filter-sterilized prior to use.

Ischemia Reperfusion Injury and Injection of Cells into the Adult Kidney Cortex

Open abdomen operation was performed on anesthetized female C57B/L6 mice at an age of 2 to 3 months. Bilateral renal pedicles were clamped with atraumatic vascular clamps

(Roboz Surgical Instrument Company) for 26 minutes of occlusion, during which time mice were kept in a warm chamber at 37° C., which was followed by reperfusion. During the occlusion, freshly sorted Flk1⁺Sca1⁺ cells or control cell populations, namely Flk1⁺Sca1⁻, Flk1⁻Sca1⁺, and Flk1⁻Sca1⁻ cells, from ROSA26 adult mouse kidney, were processed. Single cell status following sorting was confirmed by microscopy, and cells were counted using a hemocytometer. 25 μl cell suspension of 10,000 cells in normal saline was loaded into insulin syringes (½ cc lo-dose U-100 insulin syringe; Becton Dickinson) and injected 15 minutes after reperfusion into recipient mice at the lower pole of the cortex. The same amount of cells was injected for all sorted groups (Flk1⁺Sca1⁺ double positive cells and the three control groups), in addition to the same volume for a normal saline control. In sham-operated mice, the abdomen was opened, 26 minutes of occlusion of bilateral renal arteries was performed, followed by reperfusion, and normal saline was injected. Kidneys were harvested 12 days after ischemia reperfusion injury and injection of sorted cells.

Metanephros Organ Culture and Microinjection of Flk1⁺Sca1⁺ Cells into Cultured Organ Rudiments

Organ cultures were carried out essentially according to previously established protocols (Lin et al. (2001) Dev Dyn. 222: 26-39). Dissected organ rudiments were placed directly onto slices of filters (pore size 0.1 mm Nucleopore filters; Costar) supported by stainless steel grids. Suitable culture medium (DMEM; Gibco) was supplemented with 20% FCS (Gibco) and penicillin/streptomycin (GibcoBRL). Samples were incubated in 5% CO₂ in humidified air at 37° C., and culture medium was changed every other day. The cultured recipient kidneys for sorted cells were isolated from embryos at embryonic day 12.5 (E12.5) and cultured overnight (compare Steenhard et al. (2005) J Am Soc Nephrol. 16: 1623-31). Flk1⁺Sca1⁺ cells sorted freshly from ROSA26 mice, as well as control group cells, were counted, in some experiments labeled with carboxyfluoroscein succinimidyl ester (CFSE; according to the manufacturer's standard protocol, Vybrant CFDA SE Cell Tracer Kit,

Molecular Probes), and loaded into a beveled glass capillary injection pipette (World Precision Instruments) at a concentration of 60 cells/nl. Each kidney received six microinjections (2 nl each) using an IM 300 Microinjector (Narishige). Kidneys were allowed to grow in organ culture on membranes for 7 days at 37° C. and 5% CO₂.

Tissue Processing and X-gal Staining

In vivo chimera kidneys were harvested 12 days after cell injection. Kidneys were perfusion-fixed with 0.2% paraformaldehyde (PFA; Lin et al. (2003) Int J Dev Biol. 47: 3-13). After freeze protection with 30% sucrose, kidneys were embedded into suitable embedding compound (OCT compound; Sakura) and cut in 5 μm thick serial frozen sections. For in vitro cultured metanephroi, explants were either embedded in suitable resin (JB-4 resin) or OCT compound. For resin embedding, tissue was fixed in 100% MEOH at −20° C. followed by whole mount X-gal staining. X-gal staining to detect bacterial beta-galactosidase expression was carried out according to previously described protocols (Lin et al. (2001) Development 128: 1573-85; Weiss et al. (1999) Histochem J. 31: 231-6). Briefly, sections were dried, fixed in fixative with 1% PFA and 0.2% glutaraldehyde, 2 mM MgCl₂, 0.02% Na-deoxycholate, and 5 mM EGTA in PBS on ice for 10 minutes, and incubated in X-gal mixture (2 mM MgCl₂, 0.02% Na-deoxycholate, 5 mM EGTA, 5 mM K3Fe(CN)₆, 5 mM K₄Fe(CN)₆.3H2O (all Sigma-Aldrich) and 1 mg X-gal (Roche)) at a pH of 7.8 at 37° C. for 8 to 16 hours. Positive and negative control sections from Rosa26 and B57BL/6 mice were used for every X-gal staining set to judge the correct time to stop the reaction. After phosphate buffered saline (PBS) washing and an ethanol dehydration series, cultured explants were embedded in suitable resin (JB-4 resin; Electron Microscopy Research) and sectioned at a thickness of 5 μm. Slides were counterstained with nuclear fast red (Biomeda), mounted, and examined using a Nikon microscope. For frozen sections, kidney explants from organ culture were harvested 7 days after injection, fixed in freshly prepared 4% PFA, cryoprotected with 30% sucrose, and frozen in suitable embedding compound (OCT). Frozen serial sections, 5 μm thick, were postfixed in 4% PFA, rinsed twice in PBS containing 2 mM MgCl₂, and incubated in detergent rinse (0.1 M phosphate buffer at pH 7.3, 2 mM MgCl₂, 0.01% sodium deoxycholate, and 0.02% Nonidet P-40) for 10 min on ice. X-gal staining was performed overnight at 37° C. in color development solution (detergent rinse with 5 mM potassium ferricyanide, 5 mM potassium ferrocyanide, 20 mM Tris, and 1 mg/ml X-gal). Slides were postfixed in 4% PFA, dehydrated through graded ethanol, cleared in xylene, and coverslipped. In some cases, kidneys underwent color development as whole mounts before embedding (in OCT) and serial sectioning. For quantification, X-gal staining was used as an identifier of integrated tubular cells and new tubules generated from donor cells after ischemia reperfusion injury and injection into metanephroi. For the ischemia reperfusion injury model, the average number of beta-galactosidase-positive cells was determined by counting all central 100 sections in 20× fields; for the metanephroi injection model, the average tubule number was calculated from all sections by taking into account only tubules with three or more X-gal-stained cells; in both cases by using suitable imaging (Scion Image (NIH)).

Immunofluorescence

Immunofluorescence for different antibodies was performed in the following manner. After air-drying and fixation with 4% PFA, sections were treated with 1% SDS for 5 minutes followed by PBS washing twice. Sections were incubated in PBS containing 1% bovine serum albumin (BSA) for 30 minutes or with 1% serum from the animal in which the secondary antibody was raised in order to block nonspecific staining. Sections were mounted in suitable mounting media (Mounting Medium with DAPI; Vectashield, Vector Laboratories) and examined using a suitable fluorescent microscope (Nikon Eclipse E800 epifluorescence microscope). Images were captured digitally using a suitable camera and software (Hamamatsu Orca CCD camera and IPLab Spectrum software). Final images (TIFF files) were imported into a suitable graphics editing software program (Adobe Photoshop). Antibodies used were against Flk1 (IHC, BD Pharmingen); anti-mouse Sca-1/Ly6 (monoclonal, R&D system); rabbit anti-mouse aquaporins 1 and 2 (AQP1 and AQP2) for the proximal tubule and collecting duct, respectively (from Dr. Dennis Brown, Massachusetts General Hospital, Boston, Mass.); rabbit anti-mouse THP for labeling the thick ascending limb of Henle and the distal tubule (from Dr. John Hoyer, Children's Hospital of Philadelphia, Philadelphia, Pa.); and rabbit anti-mouse megalin for the brush border of the proximal tubule (Sabolic et al. (2002) Am J Physiol Renal Physiol. 283: F1389-402; from Dr. Daniel Biemesderfer, Yale University School of Medicine, New Haven, Conn.).

For immunofluorescence evaluation of beta-galactosidase expression in some experiments, primary anti-mouse biotinylated betagal antibody (Sigma) was applied to 5 μm thick serial sections, followed by secondary fluorescein-streptavidine antibody (Vector lab). Background reduction was performed with 4% PFA fixation for 10 minutes on ice, three times PBS washing, 1% SDS treatment for 5 minutes, three times PBS washing, and 15 minutes each of 3% BSA block and streptavidin/biotin block.

For Flk1/Sca1 colocalization studies by immunofluorescence, frozen sections were cut at 5 μm thickness, fixed in 4% PFA, washed three times in PBS, treated with 1% SDS for 4 minutes, washed again three times in PBS, blocked with 5% donkey serum in 3% BSA at room temperature for 30 minutes, and incubated in a hybridization chamber with primary antibodies at 4° C. overnight. Incubation with secondary antibodies, namely FITC-conjugated donkey anti-rat and texas red-conjugated donkey anti-goat (both Jackson ImmunoResearch) occurred at room temperature for 30 minutes.

The different cell populations obtained by cell sorting were analyzed by immunofluorescence after short-term culture or centrifugation to glass slides. Cells were allowed to attach to poly-L-lysine (Sigma)-pretreated chamber slides (Lab-Tek II chamber slide system, Nalge Nunc International Corporation) for 4 hours. Alternatively, cells were centrifuged at 1000 revelations per minute for 5 minutes by using a Cytospin 4 instrument (Thermo Shandon). Cells were fixed with 4% PFA on ice for 10 minutes, washed twice with PBS for 5 min, and incubated with the primary antibody at 4° C. overnight. Primary antibodies included goat anti-human vimentin antibody (Chemicon International) and mouse anti-cytokeratin 5&8 (Chemicon), both in 3% BSA and 5% donkey serum. Incubation with the secondary antibody texas red donkey anti-goat occurred at room temperature for 30 minutes. Counterstaining with DAPI-containing mounting medium (Vector Lab) was performed, and cells were photographed with a suitable microscope (Nikon). Vimentin- and cytokeratin-positive cells were evaluated by counting all positive cells in five random, non-overlapping, defined microscopic fields at 200× magnification.

Statistical Analysis

Results were analyzed to yield mean values and standard deviations for each data set. Comparisons between groups were performed using a two-tailed or unilaterally tailed paired student t test. A P value of <0.05 was considered statistically significant.

### Equivalents

From the foregoing description, it will be apparent that variations and modifications may be made to the invention described herein to adopt it to various usages and conditions. Such embodiments are also within the scope of the following claims.

### Incorporation By Reference

All patents and publications mentioned in this specification are herein incorporated by reference to the same extent as if each independent patent and publication was specifically and individually indicated to be incorporated by reference.

1. An isolated cell having tubulogenic potential, wherein the cell is Flk1 positive Sca1 positive and CD34 negative.

2. (canceled)

3. The isolated cell of claim 1, wherein the cell is derived from adult or embryonic kidney.

4. The isolated cell of claim 2, wherein the cell fails to express a polypeptide or expresses reduced levels of a polypeptide, wherein the polypeptide is selected from the group consisting of c-kit, CD34, CD45, CD31, and cytokeratin relative to a reference.

5. The isolated cell of claim 2, wherein the cell is vimentin positive.

6. An isolated adult kidney cell having tubulogenic potential, wherein the cell is Flk1 positive, Sca1 positive, vimentin positive and negative for c-kit, CD34, CD45, CD31, and cytokeratin.

7. The isolated cell of claim 6, wherein the cell is selected as Flk1 positive, Sca1 positive, vimentin positive, c-kit negative, CD34 negative, CD45 negative, CD31 negative, or cytokeratin negative using an immunoassay.

8. The isolated cell of claim 7, wherein the immunoassay is analytical flow cytometry.

9. An isolated population of cells having tubulogenic potential, wherein at least 85% of the cells are Flk1 positive Sca1 positive and CD34 negative.

10. (canceled)

11. The isolated population of claim 9, wherein the population is derived from adult or embryonic kidney.

12. The isolated population of claim 9, wherein at least 80% of the cells present in the population fail to express or express reduced levels of a polypeptide selected from the group consisting of c-kit, CD34, CD45, CD31, and cytokeratin.

13. The isolated population of claim 9, wherein at least 80% of the cells present in the population are vimentin positive.

14-42. (canceled)
